(19) United States
(12) Patent Application Publication
(10) Pub. No.: US 2007/0167511 A1
(43) Pub. Date: Jul. 19, 2007

(54) Medicinal composition for prevention or treatment of overactive bladder accompanying nervous disorder

(75) Inventors: Yoshinobu Yamazaki, Nagano (JP); Takahiro Imamura, Nagano (JP)

Correspondence Address: SUGHRUE MION, PLLC, 2100 PENNSYLVANIA AVENUE, N.W., SUITE 800, WASHINGTON, DC, 20037, US

(73) Assignee: KISSEI PHARMACEUTICAL CO,. LTD., NAGANO (JP)

(21) Appl. No.: 10/598,533

(22) PCT Filed: Feb. 23, 2005
(86) PCT No.: PCT/JP05/02913

§ 371 Date: Sep. 1, 2006

(30) Foreign Application Priority Data
Mar. 5, 2004 (JP) 2004-061476

(51) Int. Cl. A61K 31/405

(52) U.S. Cl. 514/415

(57) ABSTRACT

The present invention provides pharmaceutical compositions for the prevention or treatment of overactive bladder accompanied with neurogentic disorders. The pharmaceutical compositions comprise as an active ingredient indoline derivatives represented by the following general formula (I) or pharmaceutically acceptable salts thereof and are useful for the prevention or treatment of OAB accompanied with neurogenic disorders such as cerebrovascular disorders, Parkinson's disease, spinal cord involvement or the like.

In the formula, R represents an optionally substituted aliphaticacyl group, an optionally substituted lower alkyl group, an optionally substituted aromatic acyl group or the like; R¹ represents a cyano group or a carbamoyl group; and R² represents an optionally substituted lower alkyl group.

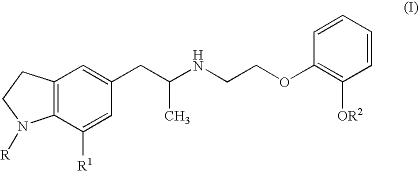

### TECHNICAL FIELD

The present invention relates to pharmaceutical compositions which are useful for the invention or treatment of overactive bladder accompanied with neurogenic disorders.

More particularly, the present invention relates to pharmaceutical compositions for the prevention or treatment of overactive bladder accompanied with neurogenic disorders, which comprises as an active ingredient an indoline derivative represented by a general formula: 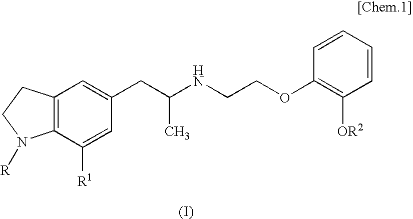
in the formula, R represents a saturated or unsaturated aliphatic acyl group which may have as a substituent a hydroxy group, a lower alkoxy group, a carboxy group a lower alkoxycarbonyl group, a cycloalkyl group, an aryl group or one or more halogen atoms; a hydroxy lower alkyl group; an aliphatic acyloxyalkyl group; a lower alkyl group which has as a substituent a lower alkoxy group, a carboxy group, a lower alkoxycarbonyl group, an aryl-substituted lower alkoxycarbonyl group, a carbamoyl group, a mono or di(lower alkyl)-substituted carbamoyl group or a cyano group; an aromatic acyl group which may have as a substituent one or more halogen atoms; a furoyl group or a pyridylcarbonyl group; R¹ represents a cyano group or a carbamoyl group; R² represents a lower alkyl group which may have as a substituent a cyano group, an aryl group or one or more halogen atoms; or a pharmaceutically acceptable salt thereof.

### BACKGROUND ART

Overactive bladder (OAB) is defined as a disease based on symptoms of urgency, usually with frequency and with or without urge incontinence. The definition of OAB is proposed in the new standardization of terminology reported by the International Continence Society (ICS) (see non-Patent Reference 1). From the epidemiologic survey conducted as a research project by Japanese Neurogenic Bladder Society, it is estimated that there are more than 8 million patients with OAB now (see non-Patent Reference 2).

OAB mostly develops accompanied with neurogenic disorder, lower urinary tract obstruction and others in addition to the ideopatic OAB without defined causes, and various therapeutic methods are performed depending on the pathogenesis (see non-Patent Reference 3). As for the OAR accompanied with neurogenic disorders, neurogenic disorder induced by origin disorder such as brain blood disorder, Parkinson's disease, spinal cord injury or the like causes the unusual micturition control, sensory disorder or the like, and that causes OAB. Thus, nevertheless many therapeutic methods are attempted to improve the urgency, frequency or the like of OAB, enough therapeutic effects are not necessarily obtained. Therefore, the establishment of the new therapeutic method suitable for each origin disorder or disease aspect has been desired.

Now, as the therapeutic methods for OAB accompanied with neurogenic disorder, combination of behavioral modification to establish the normal voiding pattern such as timed voiding training, pelvic floor muscle training or education for patients and medication is commonly used. But anticholinergic drugs mainly used in medication have the possibility of the side effects such as dry mouth, constipation, voiding dysfunction, central nervous system symptoms or the like and the therapeutic efficacy is sometimes insufficient. Therefore, the early development of new drugs that have high safety and potency has been desired (see non-Patent Reference 4).

It has been reported that the compounds represented by the above general formula (I) or a pharmaceutically acceptable salts thereof have the selective inhibitory effects against the urethral smooth muscle contraction and decrease the urethral pressure without a significant effect on the blood pressure, and they are extremely useful compounds as drugs for the treatment of the voiding dysfunction or the like induced by BPH (see Patent Reference 1). But it has been neither reported nor suggested that these compounds represented by the above general formula (I) are useful for the prevention or treatment of OAB accompanied with neurogenic disorders.

Patent Reference 1: Japanese Patent Publication

Non-patent Reference 1: Yukio Honma, et. al., [The standardization of terminology of lower urinary tract function: report from the standardization sub-committee of the international continence society], Journal of Neurogenic bladder society, 2003, Vol. 14, No. 2, pp. 278-289;

Non-patent Reference 2: Yukio Honma, et. al., [Epidemiologic survey on urination], Journal of Neurogenic bladder sociey, 2003, Vol. 14, No. 2, pp. 266-277;

Non-patent Reference 3: Osamu Yamaguchi, Mechanism of overactive bladder, PHARMACIA SCOPE, published by Pharmacia Company, 2003, Vol. 42, No. 4, pp. 12-13;

Non-patent Reference 4: Osamu Nishimura, What is overactive bladder (OAB)?, PHARMACIA SCOPE, published by Pharmacia Company, 2003, Vol. 42, No. 1, pp. 14-15.

### DISCLOSURE OF THE INVENTION

### Problem to be Solved by the Invention

The object of the present invention is to provide pharmaceutical compositions useful for the prevention or treatment of OAB accompanied with neurogenic disorders.

### Means of Solving the Problems

The present inventors have studied earnestly to find a compound useful for the prevention or treatment of OAB accompanied with neurogenic disorders, and found that a compound represented by the above general formula (I) remarkably inhibits the frequency of involuntary contraction in filling phase and has an effect to prolong the micturition interval, thereby forming the basis of the present invention.

That is, present invention relates to:

[1] a pharmaceutical composition for the prevention or treatment of overactive bladder accompanied with neurogenic disorders, which comprises as an active ingredient an indoline derivative represented by a general formula: 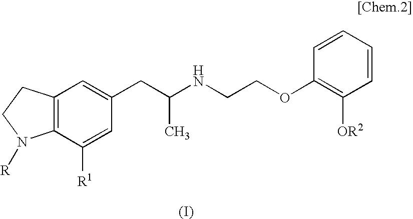
in the formula, R represents a saturated or unsaturated aliphatic acyl group which may have as a substituent a hydroxy group, a lower alkoxy group, a carboxy group, a lower alkoxycarbonyl group, a cycloalkyl group, an aryl group or one or more halogen atoms; a hydroxy lower alkyl group; an aliphatic acyloxyalkyl group; a lower alkyl group which has as a substituent a lower alkoxy group, a carboxy group, a lower alkoxycarbonyl group, an aryl-substituted lower alkoxycarbonyl group, a carbamoyl group, a mono or di (lower alkyl)-substituted carbamoyl group or a cyano group; an aromatic acyl group which may have as a substituent one or more halogen atoms; a furoyl group or a pyridylcarbonyl group; R¹ represents a cyano group or a carbamoyl group; R² represents a lower alkyl group which may have as a substituent a cyano group, an aryl group or one or more halogen atoms; or a pharmaceutically acceptable salt thereof;

[2] a pharmaceutical composition as described in the above [1] wherein the active ingredient is (−)-1-(3-hydroxypropyl)-5-((2R)-2-{[2-({2-[(2,2,2-trifluoroethyl)-oxy]phenyl}oxy)ethyl]amino}propyl)-2,3-dihydro-1H-indol-7-carboxamide or a pharmaceutically acceptable salt thereof;

[3] a pharmaceutical composition as described in the above [1] or [2] wherein the neurogenic disorder is cerebral infarction, Parkinson's disease, spinal cord involvement, peripheral neurogenic disorder or multiple sclerosis;

[4] a pharmaceutical composition as described in any of the above [1] to [3] which is used in combination with one or more other agents used for overactive bladder accompanied with neurogenic disorder;

[5] a pharmaceutical composition as described in the above [4] wherein the other agent used for overactive bladder accompanied with neurogenic disorder is an agent selected from an anticholinergic drug, an anti-anxiety drug, a cholinergic drug, a cholinesterase inhibitor, an antispasmodic drug, an anti-inflammatory drug and an antimicrobial drug; and the like.

In the present invention, the term “overactive bladder (OAB) accompanied with neurogenic disorder” means overactive bladder defined by the above new standardization of terminology of ICS, which is caused by a neurogenic disorder. As the neurogenic disorder, cerebrovascular disorders such as cerebral infraction, cerebral apoplexy and the like, parkinson's disease, spinal cord involvement such as spinal cord injury and the like, peripheral neurogenic disorders accompanied with diabetes or the like, multiple sclerosis and the like can be illustrated. The OAB accompanied with neurogenic disorders includes neurogenic bladder (for example, that caused by cerebrovascular disorders, spinal cord involvement, diabetic neuropathy, multiple sclerosis or the like) and unstable bladder, but does not include that caused by lower urinary tract obstructive diseases such as benign prostatic hypertrophy.

The present inventors confirmed that a compound represented by the above general formula (I) exerts an effect to inhibit the frequency of involuntary contraction in filling phase and an effect to prolong the micturition interval by pharmacological tests using rats with spinal cord injuries. These results support the usefulness of the present compounds for the urinary urgency and frequency in OAB accompanied with neurogenic disorder, and therefore, it has been shown that a compound represented by the above general formula (I) is extremely useful for the prevention or treatment of OAB accompanied with neurogenic disorder.

In a compound represented by the above general formula (I), the term “lower alkyl” means straight-chained or branched alkyl having 1 to 6 carbon atoms such as methyl, ethyl, propyl, isopropyl, butyl, isobutyl, sec-butyl, tert-butyl, pentyl, hexyl or the like; the term “lower alkoxy” means straight-chained or branched alkoxy having 1 to 6 carbon atoms; and the term “cycloalkyl” means 5 to 7-membered cyclic alkyl, respectively. In addition, the term “aryl” means an aromatic hydrocarbon group such as phenyl, naphthyl or the like; the term “aromatic acyl” means acyl of carboxylic acid having the above aryl; the term “aliphatic acyl which can have an unsaturated bond” means acyl of straight-chained or branched alkylcarboxylic acid having 2 to 7 carbon atoms or acyl of straight-chained or branched alkenylcarboxylic acid having 3 to 7 carbon atoms; and the term “aliphatic acyloxyalkyl” means the above lower alkyl having a hydroxy group substituted by the above aliphatic acyl group having 4 to 13 carbon atoms, respectively. Furthermore, the term “furoyl” means 2-furoyl or 3-furoyl; the term “pyridylcarbonyl” means 2-pyridylcarbonyl, 3-pyridylcarbonyl or 4-pyridylcarbonyl; and the term “halogen atom” means a fluorine atom, a chlorine atom, a bromine atom or an iodine atom, respectively.

In compounds represented by the above general formula (I), as a preferable compound, for example, (−)-1-(3-hydroxypropyl)-5-((2R)-2-{[2-({2-[(2,2,2-trifluoroethyl)oxy]phenyl}oxy)eth yl]amino}propyl)-2,3-dihydro-1H-indol-7-yl-carboxamide and pharmaceutically acceptable salts thereof (among them, a dihydrobromide is hereinafter referred to as “compound 1”) can be illustrated.

Several manufacturing methods of the compounds represented by the above general formula (I) are known, and they can be easily prepared in a method described in literatures or the like (see the above Patent Reference 1).

As the pharmaceutically acceptable salt thereof, a salt with an inorganic base such as sodium, potassium, calcium or the like, a salt with an organic amine such as morpholine, piperidine or the like, a salt with a mineral acid such as hydrochloric acid, hydrobromic acid, sulfuric acid or the like, a salt with an organic acid such as methanesulfonic acid, benzenesulfonic acid, p-toluenesulfonic acid, acetic acid, citric acid, succinic acid, tartaric acid, 2,4-dimethyl-benzenesulfonic acid, 2,5-dimethylbenzenesulfonic acid, 2,4,6-trimethylbenzenesulfonic acid, (+)-camphorsulfonic acid, (−)-camphorsulfonic acid, 4-chlorobenzenesulfonic acid, 2-naphthalenesulfonic acid, 1-butanesulfonic acid, fumaric acid, glutamic acid aspartic acid and the like can be illustrated.

The compounds represented by the above general formula (I) include their hydrates and solvates with pharmaceutically acceptable solvents such as ethanol or the like. In addition, the compounds of the present invention include both of amorphous forms and crystal forms, and a single polymorphic, a mixture of two or more polymorphs and a mixture of a polymorph and an amorphous form thereof.

A compound represented by the above general formula (I) has at least one asymmetric carbon atom, and therefore there are two configurations, R-configuration and S-configuration, for each asymmetric carbon. In the present invention, a compound with either configuration can be employed, and a mixture thereof can be also employed.

Of the compounds represented by the above general formula (I), there are geometrical isomers, E and Z-isomers, in each compound having an unsaturated bond. In the present invention, either of the isomers can be employed.

Furthermore, the compounds represented by the above general formula (I) can be also used in combination with one or more other drugs used for OAB accompanied with neurogenic disorder. Examples of the other drugs which can be used in combination with include, for example, an anticholinergic drug (tolterodine, oxybutynin, propiverine or the like), an anti-anxiety drug, a cholinergic drug (bethanechol chloride or the like), a cholinesterase inhibitor (distigmine bromide or the like), an antispasmodic drug (flavoxate or the like), an anti-inflammatory drug and an antimicrobial drug and the like.

In the case of uses of the compound represented by the above general formula (I) in combination with the above one or more other drugs, the present invention includes either dosage forms of simultaneous administration as a single preparation or separated preparations in way of the same or different administration route, and administration at different dosage intervals as separated preparations in way of the same or different administration route. A pharmaceutical combination comprising the compound of the present invention and the above other drug(s) includes both dosage forms as a single preparation and separated preparations for combination as mentioned above.

The compounds represented by the above general formula (I) can obtain more advantageous effects than additive effects in the prevention or treatment of the above diseases when using suitably in combination with the above one or more other drugs. Also, the administration dose can be decreased in comparison with administration of either drug alone, or adverse effects of the above drugs coadministrated can be avoided or declined.

When the pharmaceutical compositions of the present invention are employed in the practical treatment, various dosage forms are used depending on their uses. As examples of the dosage forms, powders, granules, fine granules, dry syrups, tablets, capsules, injections, solutions, ointments, suppositories, poultices and the like are illustrated, which are orally or parenterally administered.

These pharmaceutical compositions can be prepared by suitably admixing with or by diluting and dissolving with an appropriate pharmaceutical additive such as excipients, disintegrators, binders, lubricants, diluents, buffers, isotonicities, antiseptics, moistening agents, emulsifiers, dispersing agents, stabilizing agents, dissolving aids and the like, and formulating the mixture in accordance with conventional methods. In the case of the uses in combination with other drug(s), they can be prepared by formulating each active ingredient together or individually in a similar manner as defined above.

For example, powders can be formulated by, if desired, admixing well a compound represented by the above general formula (I) with appropriate excipients, lubricants and the like.

For example, tablets can be formulated by, if desired, admixing a compound represented by the above general formula (I) with appropriate excipients, disintegrating agents, binders, lubricants and the like, and compressing the mixture in accordance with conventional methods. The tablets, further if desired, can be suitably coated to provide film-coated tablets, sugar-coated tablets, enteric-coated tablets and the like.

For example, capsules can be formulated by, if desired, admixing well a compound represented by the above general formula (I) with appropriate excipients, lubricants and the like, or formulating granules or fine-powders in accordance with conventional methods, and then filling the compositions in appropriate capsules.

When the pharmaceutical compositions of the present invention are employed in the practical treatment, the dosage of a compound represented by the above general formula as the active ingredient is appropriately decided depending on the body weight, age, sex and degree of diseases or treatment of each patient, which is approximately within the range of from 0.5 to 500 mg per day per adult human in the case of oral administration and approximately within the range of from 0.05 to 100 mg per day per adult human in the case of parenteral administration, and the daily dose can be divided into one to several doses per day and administered. Also, in the case of the uses in combination with the above other drug(s), the dosage of the compound of the present invention can be decreased depending on the dosage of the other drug(s).

### EFFECT OF THE INVENTION

The pharmaceutical compositions of the present invention exert an excellent improving effect on frequency of involuntary contraction and micturition interval as indicators of urinary urgency and frequency in OAB accompanied with neurogenic disorder such as spinal cord injury or the like. The present invention can provide a pharmaceutical composition useful for the prevention or treatment of OAB accompanied with neurogenic disorder.

### BRIEF DESCRIPTION OF DRAWINGS

FIG. 1 shows the effects on the micturition interval in rat spinal cord injured OAB model. The “□” shows data before drug administration and the “▪” shows data on compound 1. The vertical axis indicates the percentage of the micturition interval against the value before drug administration.

FIG. 2 shows the effects of the compound on the frequency of involuntary contraction in rat spinal cord injured OAB model. The “□” shows data before drug administration and the “▪” shows data on compound 1. The vertical axis indicates the percentage of the frequency of involuntary contraction against the value before drug administration.

### BEST MODE TO PRACTICE THE INVENTION

The present invention is further illustrated in more detail by way of the following Example.

### EXAMPLE 1

Efficacy on Urodynamic Study in Rat Spinal Cord Injured OAB Model

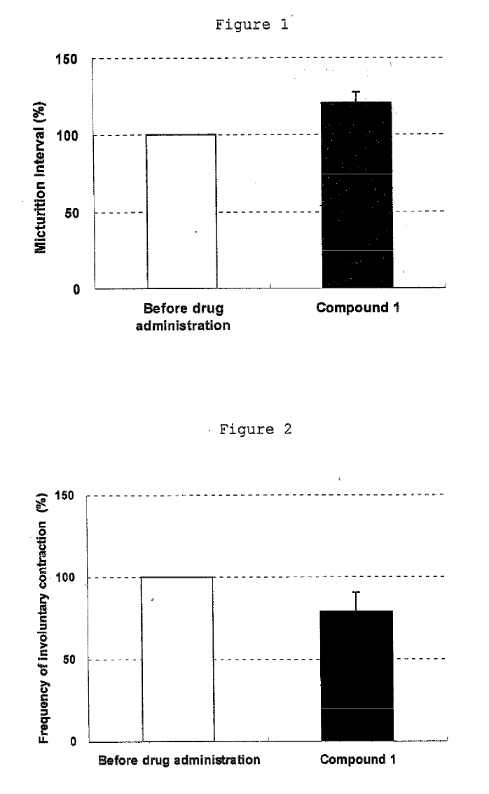

In ether anesthetized female rats, spinal cord transection was performed at the level of Th10. About 1 month after the spinal cord transection, each rat was anesthetized with pentobarbital and a catheter filled with saline was implanted into the urinary bladder, ligated, secured on the back of the neck and closed. Seven days after the bladder catheter implantation, another catheter filled with heparin-containing saline was implanted into the carotid vein, and ligated, secured on the back of the neck and closed. The next day, cystometry was performed in the conscious rat under free moving. Saline was instilled into the urinary bladder at a rate of 12 mL/hr. A drug was injected through the carotid vein catheter that was secured on the back of the neck. As a result, in this female rat spinal cord injured model, involuntary contractions were observed in filling phase. Intravenous injection of compound 1 (0.1 mg/kg) prolonged the micturition interval by about 20% (FIG. 1) and decreased the frequency of involuntary contraction in filling phase by 20% (FIG. 2) in the same model.

As mentioned above, it was shown that the compounds represented by the above general formula (I) exert an excellent improving effect on the frequency of the involuntary contraction and micturition interval as the parameter of urgency and frequency, respectively, in spinal cord injured OAB model, extremely useful for the prevention or treatment of OAB associated with neurogenic disorders.

### INDUSTRIAL APPLICABILITY

The pharmaceutical compositions of the present invention exert an excellent improving effects on urinary urgency and frequency and are extremely useful as an agent for the prevention or treatment of OAB accompanied with neurogenic disorders.

1. A method for the prevention or treatment of overactive bladder accompanied with neurogenic disorders, which comprises administering an effective amount of an indoline derivative represented by a formula: 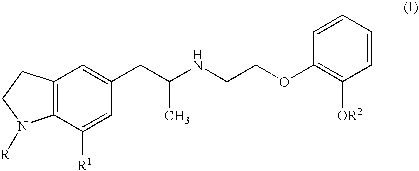
wherein in the formula, R represents a saturated or unsaturated aliphatic acyl group which may have as a substituent a hydroxy group, a lower alkoxy group, a carboxy group, a lower alkoxycarbonyl group, a cycloalkyl group, an aryl group or one or more halogen atoms; a hydroxy lower alkyl group; an aliphatic acyloxyalkyl group; a lower alkyl group which has as a substituent a lower alkoxy group, a carboxy group, a lower alkoxycarbonyl group, an aryl-substituted lower alkoxycardonyl group, a carboarloyl group, a mono or di (lower alkyl)-substituted carbarnoyl group or a cyano group; an aromatic acyl group which may have as a substituent one or more halogen atoms; a furoyl group or a pyridylcarbonyl group; R¹ represents a cyano group or a carbamoyl group; R² represents a lower alkyl group which may have as a substituent a cyano group, an aryl group or one or more halogen atoms; or a pharmaceutically acceptable salt thereof.

2. A method for the prevention or treatment as claimed in claim 1, which comprises administering an effective amount of (-)-1-(3-hydroxypropyl)-5-((2R)-2-{[2-({2-[(2,2,2-trifluoro-ethyl)oxy]phenyl}oxy)ethyl]amino}propyl)-2,3-dihydro-1H-indol-7-carboxamide or a pharmaceutically acceptable salt thereof.

3. A method for the prevention or treatment as claimed in claim 1, wherein the neurogenic disorder is cerebral infarction, Parkinson's disease, spinal cord involvement, peripheral neurogenic disorder or multiple sclerosis.

4. A method for the prevention or treatment as claimed in claims 1, which comprises administering in combination with one or more other agents used for overactive bladder accompanied with neurogenic disorder.

5. A method for the prevention or treatment as claimed in claim 4 wherein the other agent used for overactive bladder accompanied with neurogenic disorder is an agent selected from an anticholinergic drug, an anti-anxiety drug, a cholinergic drug, a cholinesterase inhibitor, an antispasmodic drug, an anti-inflammatory drug or an antimicrobial drug.

6. A method for the prevention or treatment as claimed in claim 2, wherein the neurogenic disorder is cerebral infarction, Parkinson's disease, spinal cord involvement, peripheral neurogenic disorder or multiple sclerosis.

7. A method for the prevention or treatment as claimed in claim 2, which comprises administering in combination with one or more other agents used for overactive bladder accompanied with neurogenic disorder.

8. A method for the prevention or treatment as claimed in claim 3, which comprises administering in combination with one or more other agents used for overactive bladder accompanied with neurogenic disorder.

9. A method for the prevention or treatment as claimed in claim 6, which comprises administering in combination with one or more other agents used for overactive bladder accompanied with neurogenic disorder.

10. A method for the prevention or treatment as claimed in claim 7 wherein the other agent used for overactive bladder accompanied with neurogenic disorder is an agent selected from an anticholinergic drug, an anti-anxiety drug, a cholinergic drug, a cholinesterase inhibitor, an antispasmodic drug, an anti-inflammatory drug or an antimicrobial drug.

11. A method for the prevention or treatment as claimed in claim 8 wherein the other agent used for overactive bladder accompanied with neurogenic disorder is an agent selected from an anticholinergic drug, an anti-anxiety drug, a cholinergic drug, a cholinesterase inhibitor, an antispasmodic drug, an anti-inflamatory drug or an antimicrobial drug.

12. A method for the prevention or treatment as claimed in claim 9 wherein the other agent used for overactive bladder accompanied with neurogenic disorder is an agent selected from an anticholinergic drug, an anti-anxiety drug, a cholinergic drug, a cholinesterase inhibitor, an antispasmodic drug, an anti-inflammatory drug or an antimicrobial drug.
